(19) United States
(12) Patent Application Publication
(10) Pub. No.: US 2015/0007390 A1
(43) Pub. Date: Jan. 8, 2015

(54) Patient Transport System

(71) Applicants: Sultan Haider, Erlangen (DE); Ludwig Kreischer, Dormitz (DE)

(72) Inventors: Sultan Haider, Erlangen (DE); Ludwig Kreischer, Dormitz (DE)

(21) Appl. No.: 14/324,137

(22) Filed: Jul. 4, 2014

(30) Foreign Application Priority Data
Jul. 5, 2013 (DE) DE 102013213213.1

(51) Int. Cl. A61G 1/04; G01S 13/86; A61B 6/04

(52) U.S. Cl. CPC A61G 1/04; A61B 6/0407; G01S 13/862; G01S 13/865; G01S 13/867
USPC 5/600

(57) ABSTRACT

A patient transport system includes a patient transport device configured to be docked on a medical instrument, and a detection device configured to determine information relevant to an approach, occurring during docking, of the patient transport device to the medical instrument. The patient transport system assists the docking procedure of the patient transport device on the medical instrument.

This application claims the benefit of DE 10 2013 213 213.1, filed on Jul. 5, 2013, which is hereby incorporated by reference in its entirety.

### BACKGROUND

The present embodiments relate to a patient transport system, a patient transport device and a medical instrument, configured for docking of the patient transport device.

In hospitals, patients are to be transported between different diagnostic and therapy instruments, or between holding areas and examination rooms. In so doing, due to the health of the patient, the patient may be conveyed in a lying position on a patient transport device (e.g., a trolley). For diagnosis or treatment with a diagnostic or treatment instrument (e.g., computed tomography scanner, magnetic resonance imaging scanner, x-ray instrument, radiation therapy instrument, etc.), the patient is to be repositioned from the patient transport device to a space provided for the diagnosis or treatment procedure. This repositioning should be associated with as little exertion for the patient as possible. This applies, for example, to patients in a poor physical state. Current patient transport devices are, in part, configured for docking onto a medical instrument or a medical modality (e.g., the patient transport device is fastened to the medical instrument in order thus to simplify the repositioning of the patient).

Where possible, a patient transport device may dock quickly and efficiently onto a medical instrument so as to keep the patient stress low and not to interrupt the medical workflow. The document US 20060167356 A1 discloses a trolley that provides automatic assistance for the docking procedure. Sensors support the locking of the trolley on a medical instrument in order thus to avoid difficulties for the operating staff when connecting trolley and medical instrument.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, assistance for an operating staff during patient transport using a patient transport device is provided.

According to one embodiment, a patient transport system configured for a simplified docking procedure is provided. The transport system includes a patient transport device or a trolley. The patient transport device or the trolley is configured to be docked on a medical instrument. The simplification includes assisting the approach of the patient transport device within the process of docking on a medical instrument. Within the scope of one or more of the present embodiments, information relevant to this approach is determined by a detection device or measurement device. This information may be a distance between the patient transport device and the medical instrument, a speed of the patient transport device or presence of an object between the patient transport device and the medical instrument. In the process, a plurality of the aforementioned items of information may be determined within the scope of checking the region between the patient transport device and the medical instrument.

For determining the relevant information, the detection device may use, for example, ultrasound, capacitive induction, RFID technology or a camera system. A combination of these various technologies may also be used. Specifically, the detection device may be formed with an RFID transmitter/receiver pair, an optical marker and a camera, an ultrasound transmitter/receiver pair, or a combination thereof. The aforementioned elements are arranged on the patient transport device or on the medical instrument. The following options may be provided. The medical device may include at least one camera (e.g., a plurality of cameras), and the patient transport device includes an optical marker (e.g., a plurality of optical markers), or an interchanged arrangement of camera and markers. The medical device may include an RFID transmitter (e.g., a plurality of RFID transmitters), and the patient transport device includes an RFID receiver (e.g., a plurality of RFID receivers), or RFID transmitters and receivers with an interchanged arrangement. The patient transport device may include an ultrasound transmitter and ultrasound receiver (e.g., arranged in spatial proximity), where the ultrasound receiver detects ultrasonic waves reflected by the medical instrument or another object.

The aforementioned embodiments may also be used in combination.

In accordance with one development, the patient transport system includes a monitoring unit for evaluating the information determined by the detection device. The patient transport system may be configured for wireless transmission, from the patient transport device and/or the medical instrument to the monitoring unit. The patient transport system may transmit information relevant to the approach or information required for determining the information relevant to the approach. The monitoring unit may also take on, at least in part, the determination or calculation of the relevant information (e.g., be part of the detection device with respect to these objects). By way of example, the detection device may then be realized with sensor devices that are arranged on the patient transport device and/or the medical instrument and include radio transmission devices and may include calculation devices for determining the relevant information (e.g., distance, speed, presence of an interfering object).

In accordance with one development, the patient transport system includes a protection device, by which an optical or acoustic warning signal is emitted, or automatic braking of the patient transport device is brought about. These measures are adopted if the information relevant to the approach lies outside of an admissible value range or if the presence of an object between the patient transport device and the medical instrument is detected.

One or more of the present embodiments enable a patient transport device to avoid collisions (e.g., with an object situated between the device and the medical instrument or because the too high speed of approach of the patient transport device would lead to a collision with the medical instrument or to an impact on the medical instrument with risk for the mechanical parts present (on the docking site)).

### BRIEF DESCRIPTION OF THE DRAWINGS

FIG. 1 shows a schematic illustration of a situation when a patient table approaches an MRI device;

FIG. 2 shows one embodiment including cameras and optical markers;

FIG. 3 shows one embodiment including RFID transmitters and RFID receivers; and

FIG. 4 shows one embodiment using ultrasound technology.

### DETAILED DESCRIPTION

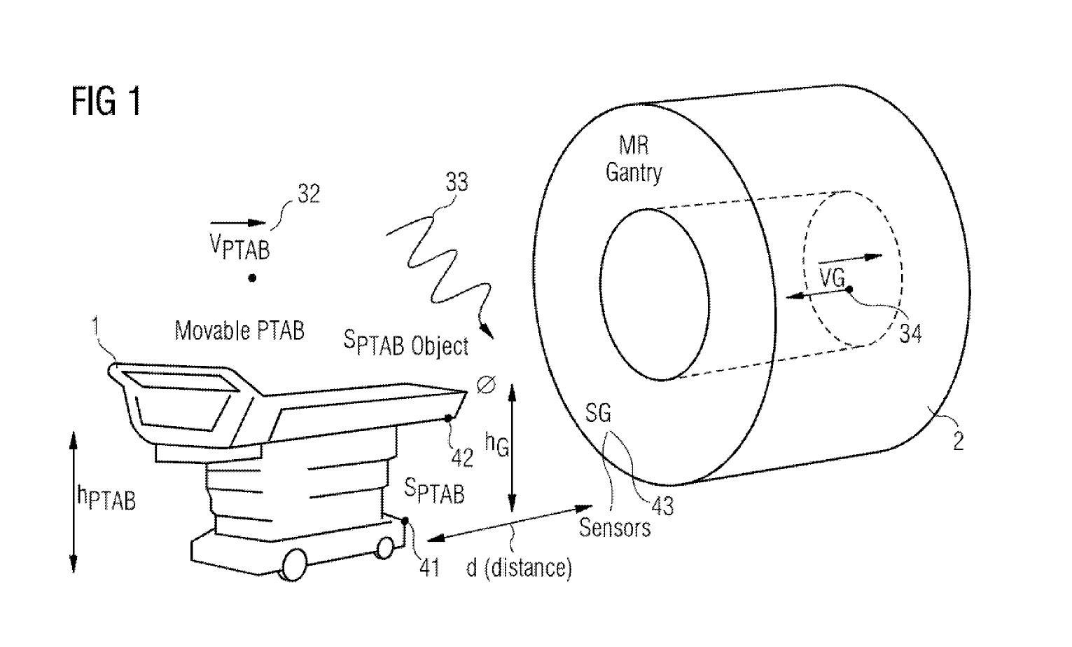

FIG. 1 shows one embodiment of a patient transport device or a trolley 1 and a medical instrument (e.g., a magnetic resonance imaging scanner 2). Two sensors 41 and 42 arranged on the patient transport device 1 have been shown. The two sensors 41 and 42 are situated directly above wheels (e.g., sensor 41) or level with the tabletop (e.g., sensor 42). If there are a plurality of sensors on the patient transport device 1, the plurality of sensors may be arranged as far apart as possible or on protruding parts so as to assume a more suitable position for collision protection. In the image, a sensor 43 is also arranged on the gantry 2. A sensor system that renders it possible to establish information relevant to the approach is thus implemented. By way of example, this information is a distance 31 between the patient transport system 1 and the medical instrument 2, a speed 32 of the patient transport system, or presence of an object between the patient transport system and the medical instrument (e.g., indicated by the arrow 33). The medical instrument may be moved for the docking procedure (e.g., to have a speed 34). In this case, either both speeds or a relative speed may be established in order to be able to initiate braking required for the docking in good time.

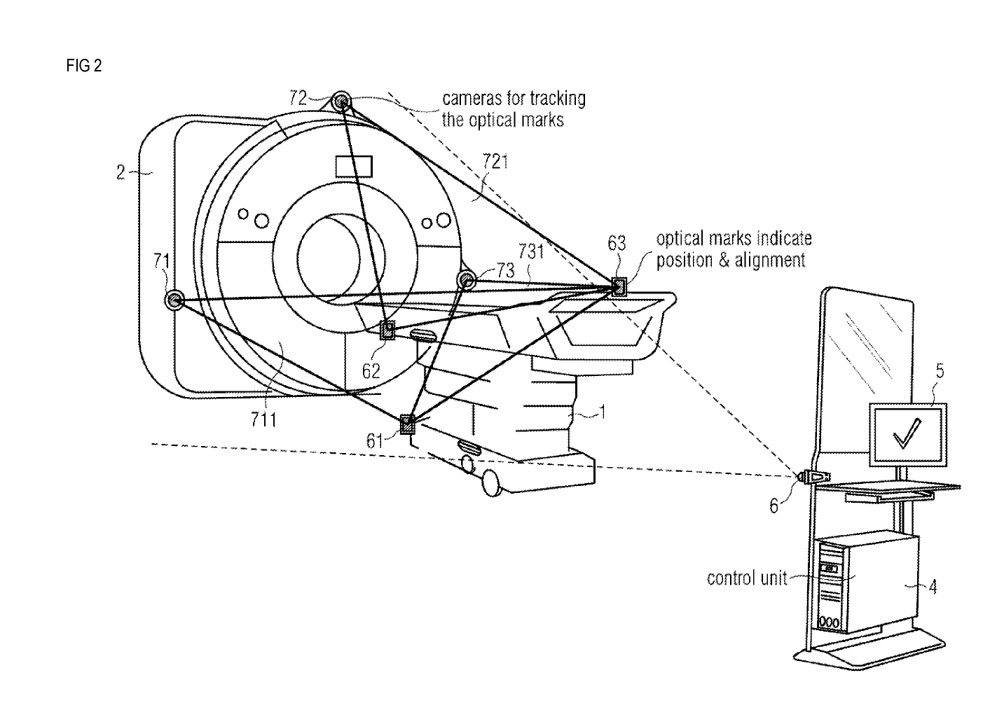

FIG. 2 shows an implementation using cameras and optical markers. Three markers 61, 62 and 63 attached to the patient transport device 1 are shown. The optical markers may be spaced apart as far as possible and provided at endpoints or possible collision points so as to be able to be detected in an improved manner and therefore be able to implement collision protection in an improved manner. For example, the marker 61 is arranged at a front, just above the wheels, while the marker 62 has been placed front left in a region of a tabletop. The marker 63 has been placed at a highest point on a rear end of the trolley 1.

The counterparts thereto are cameras 71, 72 and 73 attached to the gantry 2 of the magnetic resonance imaging scanner. The cameras 71, 72, and 73 are placed on edge regions (e.g., 71 and 73 to the center right and left; 72 directly above the gantry) so as to be able to cover a region that is as broad as possible. For the cameras 71, 72 and 73, recording regions 711, 721 and 731 have been shown in each case. These indicate that a plurality of markers may be registered with the cameras in each case. The desired information is calculated from the recorded images. It is also possible, for example, to deduce from the marker being covered that an interfering object is situated in an interspace between trolley 1 and gantry 2. The recorded information is transmitted by radio to the receiver 6 and, from there, forwarded to a monitoring unit 4. The monitoring unit 4 evaluates the received information and provides a report about a result of the evaluation. The report is displayed, for example, by the monitor 5. By way of example, a warning may be displayed at the monitor 5. The warning provides information about an impending collision. Alternatively, or parallel thereto, there may be an acoustic warning The arrangement of markers on the trolley 1 and cameras on the gantry 2, shown in this exemplary embodiment, may also be interchanged (e.g., the cameras may also be arranged on the trolley 1, while the markers may be found on the gantry 2). In one embodiment, both trolley 1 and gantry 2 may have markers and cameras in order thereby to realize a camera/marker system optimized for collision protection.

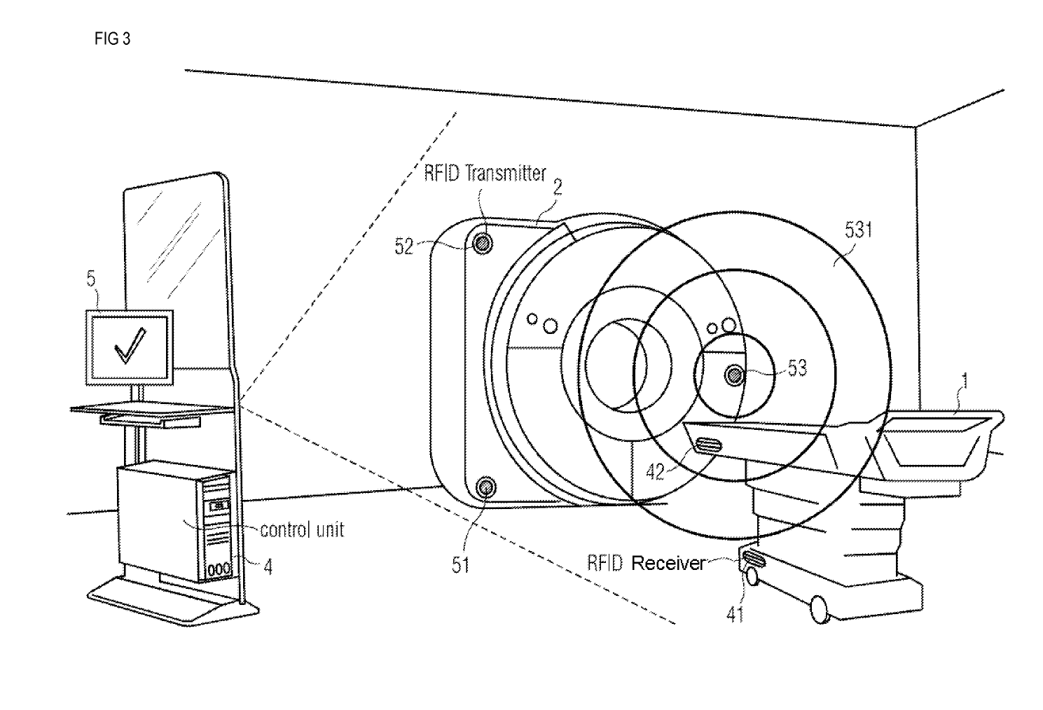

FIG. 3 shows a solution implemented using RFID transmitters or emitters and RFID sensors or receivers. Two RFID sensors 41 and 42, which are arranged on the trolley 1, are shown. The two sensors 41 and 42 are arranged top and bottom on the left-hand side of the trolley. In one embodiment, the trolley 1 also has sensors on the other side, symmetrically with respect to the sensors 41 and 42 in order to complete the sensor system. The gantry 2 has RFID transmitters. Two such transmitters 51 and 52 are shown in the image. One of the transmitters 51 is arranged near the floor, and the other one of the transmitter 52 is arranged at the upper end of the gantry in order, as a result of this spacing, to be able to cover a wider range. A third RFID transmitter 53 is arranged centrally on the other side of the gantry 2. The radiation 531 emanating from this transmitter is indicated. The radiation 531 is detected by the sensors on the patient transport device 1. Detected information is transmitted to the monitoring device 4 by radio transmission. A screen 5, by which optical feedback with respect to the evaluation result by the monitoring unit 4 may be provided, is provided. In this case, transmitters and sensors may also be interchanged in terms of positioning on the trolley 1 or gantry 2, or a system of transmitters and sensors, where both elements, the trolley 1 and the gantry 2, have both sensors and transmitters, is provided.

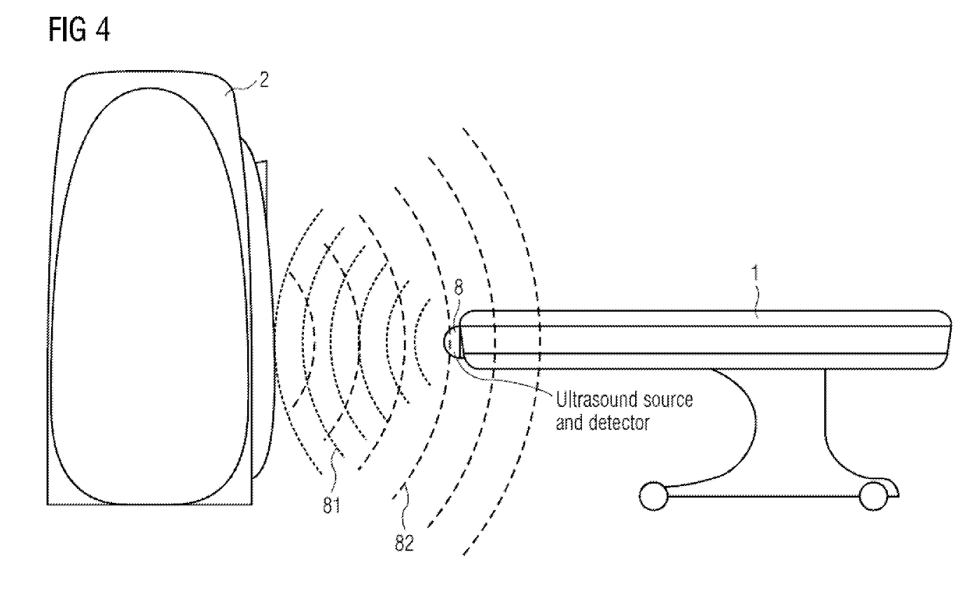

FIG. 4 depicts a further implementation. The trolley 1 has an ultrasound unit 8 that includes both an ultrasound source and an ultrasound detector. Using this unit, ultrasonic waves 81 are emitted in the direction of the gantry 2 and waves 82 are detected again after reflection by the gantry 2. In this manner, an efficient measurement of spacing and speed of the trolley 1 may be realized, and an object possibly situated between the trolley 1 and the gantry 2 may be detected via the reflected waves being evaluated.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A patient transport system comprising:
a patient transport device configured to be docked on a medical instrument; and
a detection device configured to determine information relevant to an approach occurring during docking of the patient transport device to the medical instrument.

2. The patient transport system of claim 1, wherein the relevant information comprises a distance between the patient transport device and the medical instrument, a speed of the patient transport device, or presence of an object between the patient transport device and the medical instrument.

3. The patient transport system of claim 1, wherein the detection device uses ultrasound, capacitive induction, RFID technology or a camera system for determining the relevant information.

4. The patient transport system of claim 1, wherein the patient transport system comprises a monitoring unit for evaluating the information determined by the detection device.

5. The patient transport system of claim 4, wherein the patient transport system is configured for wireless transmission, from the patient transport device, the medical instrument, or the patient transport device and the medical instrument to the monitoring unit, of the information relevant to the approach or information required for determining the information relevant to the approach.

6. The patient transport system of claim 1, wherein the detection device is formed with an RFID transmitter/receiver pair, an optical marker and a camera, an ultrasound transmitter/receiver pair, or a combination thereof, each of the RFID transmitter/receiver pair, the optical marker and the camera, the ultrasound transmitter/receiver pair, or the combination thereof being arranged on the patient transport device or on the medical instrument.

7. The patient transport system of claim 1, wherein the patient transport system comprises a protection device, by which an optical or acoustic warning signal is emitted or automatic braking of the patient transport device is brought about when the information relevant to the approach lies outside of an admissible value range or when presence of an object between the patient transport device and the medical instrument is detected.

8. The patient transport system of claim 2, wherein the detection device uses ultrasound, capacitive induction, RFID technology or a camera system for determining the relevant information.

9. The patient transport system of claim 2, wherein the patient transport system comprises a monitoring unit for evaluating the information determined by the detection device.

10. The patient transport system of claim 3, wherein the patient transport system comprises a monitoring unit for evaluating the information determined by the detection device.

11. The patient transport system of claim 2, wherein the detection device is formed with an RFID transmitter/receiver pair, an optical marker and a camera, an ultrasound transmitter/receiver pair, or a combination thereof, each of the RFID transmitter/receiver pair, the optical marker and the camera, the ultrasound transmitter/receiver pair, or the combination thereof being arranged on the patient transport device or on the medical instrument.

12. The patient transport system of claim 3, wherein the detection device is formed with an RFID transmitter/receiver pair, an optical marker and a camera, an ultrasound transmitter/receiver pair, or a combination thereof, each of the RFID transmitter/receiver pair, the optical marker and the camera, the ultrasound transmitter/receiver pair, or the combination thereof being arranged on the patient transport device or on the medical instrument.

13. The patient transport system of claim 5, wherein the detection device is formed with an RFID transmitter/receiver pair, an optical marker and a camera, an ultrasound transmitter/receiver pair, or a combination thereof, each of the RFID transmitter/receiver pair, the optical marker and the camera, the ultrasound transmitter/receiver pair, or the combination thereof being arranged on the patient transport device or on the medical instrument.

14. The patient transport system of claim 2, wherein the patient transport system comprises a protection device, by which an optical or acoustic warning signal is emitted or automatic braking of the patient transport device is brought about when the information relevant to the approach lies outside of an admissible value range or when presence of an object between the patient transport device and the medical instrument is detected.

15. The patient transport system of claim 3, wherein the patient transport system comprises a protection device, by which an optical or acoustic warning signal is emitted or automatic braking of the patient transport device is brought about when the information relevant to the approach lies outside of an admissible value range or when presence of an object between the patient transport device and the medical instrument is detected.

16. The patient transport system of claim 5, wherein the patient transport system comprises a protection device, by which an optical or acoustic warning signal is emitted or automatic braking of the patient transport device is brought about when the information relevant to the approach lies outside of an admissible value range or when presence of an object between the patient transport device and the medical instrument is detected.

17. A patient transport device for a patient transport system comprising a detection device configured to determine information relevant to an approach occurring during docking of the patient transport device to a medical instrument, the patient transport device being configured for docking on the medical instrument, the patient transport device comprising:
an optical marker, an RFID sensor, an ultrasound sensor, a capacitive sensor or a combination thereof.

18. The patient transport device of claim 17, further comprising an ultrasound source.

19. The patient transport device of claim 17, further comprising a camera or an RFID receiver.

20. A medical instrument configured for docking a patient transport device for a patient transport system, the patient transport system comprising a detection device configured to determine information relevant to an approach occurring during docking of the patient transport device to the medical instrument, the patient transport device being configured for docking on the medical instrument and comprising an optical marker, an RFID sensor, an ultrasound sensor, a capacitive sensor or a combination thereof, the medical instrument comprising:
a camera, an RFID receiver, an optical marker, an RFID sensor, an ultrasound sensor, a capacitive sensor or a combination thereof.
